(19) United States
(12) Patent Application Publication
(10) Pub. No.: US 2023/0279100 A1
(43) Pub. Date: Sep. 7, 2023

(54) Methods of Prevention or Treatment of Alzheimer's Disease by Blocking or Decreasing STING Signaling Activation

(71) Applicant: Feng Xue, McKinney, TX (US)

(72) Inventor: Feng Xue, McKinney, TX (US)

(21) Appl. No.: 17/742,375

(22) Filed: May 11, 2022

Related U.S. Application Data
(60) Provisional application No. 63/315,980, filed on Mar. 3, 2022.

(51) Int. Cl. C07K 16/28; C12N 15/113; C12N 9/22; C12N 15/11; A61P 25/28

(52) U.S. Cl. CPC C07K 16/28; C12N 15/1138; C12N 9/22; C12N 15/11; A61P 25/28; C12N 2310/141; C12N 2310/14; C12N 2310/11

(57) ABSTRACT

The disclosed embodiments relate the general methods for prevention or treatment of Alzheimer's Disease (AD) by blocking or decreasing STING (TMEM173) signaling activation. Our study showed that, in the young (2-3 months old) AD 5XFAD animal models, before the plaque accumulation of Aβ amyloid protein, blocking (e.g.,. gene knockout) or decreasing (e.g., small molecule antagonist treatment) Sting (Tmem173) signaling activation significantly reduced the pro-inflammatory activation and the proliferation of microglia (Mg) cells, enhanced the lysosomal synthesis and the protein antigen degradation of Mg cells, decreased the neuron loss and the AD neuroinflammation, and improved the cognitive ability of the AD 5XFAD mouse models. Therefore, in the mild AD or in the early stages of AD, blocking or decreasing STING signaling activation can be used for prevention or treatment of AD.

### RELATED APPLICATIONS

The present application claims priority to and is a non-provisional of U.S. Provisional Application Ser. No. 63/315,980 filed Mar. 3, 2022, all of which is incorporated herein by reference.

### TECHNICAL FIELD

The invention relates the technical field of drug research for prevention or treatment of Alzheimer's Disease (AD), in particular the application of blocking or decreasing STING (TMEM173) signaling activation in prevention or treatment of AD.

### BACKGROUND

Alzheimer's Disease (AD) is a degenerative disease of central nervous system characterized by progressive cognitive dysfunction and behavioral impairment. According to the 2015 World Alzheimer's Report, there are about 46.8 million people with AD worldwide, with an average of one new case occurring every three seconds. It is predicted that the number of people with AD will exceed 131.5 million in 2050.

AD is a central neurodegenerative disease with high mortality. In recent years, its incidence rate has been increasing rapidly, which has caused serious social burden. Chronic neuroinflammation is an important pathological feature of AD. Reducing neuroinflammation can significantly decrease the pathological damage of AD, so it is a good drug target for AD.

In the severe AD or in the late stage of AD, microglia (Mg) cells show cellular senescence. Therefore, pro-inflammatory activation of Mg can promote the degradation of Aβ amyloid plaques and reduce neuroinflammation. Besides, in the mild AD or in the early stage of AD, early neuroinflammation was also revealed in recent clinical and animal model studies (1). Our study showed that, in the young (2-3 months old) AD animal models, before the plaque accumulation of Aβ amyloid protein (2), blocking (eg. gene knockout) or decreasing (eg. small molecule antagonist treatment) Sting signaling activation significantly reduced the pro-inflammatory activation and the proliferation of Mg cells, enhanced the lysosomal synthesis and the protein antigen degradation of Mg cells, decreased the neuron loss and the AD neuroinflammation, and improved the cognitive ability of AD mouse models.

Human STING/Mouse Sting (Human TMEM173/Mouse Tmem173) signaling pathway was firstly defined as an early immune signaling pathway that activates antiviral Interferon I (IFN I) in the early stage of viral infection (3). Recent studies showed that IFN I is one of the upstream pro-inflammatory immune signaling pathways in AD chronic neuroinflammation, and can directly induce Mg to engulf synapses of neurons (4, 5). Our new study revealed that Sting signaling pathway was one of the upstream synthesis pathways of IFN I in AD chronic neuroinflammation, and was the main Mg pro-inflammatory immune signaling pathway in the mild AD before the accumulation of Aβ amyloid plaques. Therefore, STING signaling pathway is a drug target for AD.

### SUMMARY

The invention discloses an application of blocking or decreasing STING (TMEM173) signaling activation in prevention or treatment of Alzheimer's Disease (AD).

Blocking or decreasing STING signaling activation in the preparation of products for prevention or treatment of AD. The AD can be mild, moderate or severe.

STING signaling pathway refers to either of the cGAS-STING-TBK1-IRF3-IFN I cascade signaling pathway or the cGAS-STING-IKK-NFκB-IFN I cascade signaling pathway or both.

The methods for blocking or decreasing STING signaling activation refers to antibodies, siRNA, miRNA, antisense oligonucleotides, CRISPR/Cas9 gene editing products, antagonists, blockers, small molecule compounds that inhibit the function and/or activity of main molecules of STING signaling pathway, and one of or a combination of promoter elements or expression vectors, which can downregulate the gene expression levels, the mRNA expression levels, or the protein expression levels of the major molecules of STING signaling pathway.

The symptoms of blocking or decreasing STING signaling activation includes one or more of the following groups: the decreased early pro-inflammatory activation of Mg cells, the decreased proliferation of the pro-inflammatory Mg cells, the enhanced lysosomal synthesis and the protein antigen degradation of Mg cells, the decreased neuron loss, the decreased AD neuroinflammation, and the improved cognitive ability.

Blocking or decreasing STING signaling activation targets human STING signaling pathway.

The products include medicine, food, and health care products and their compositions.

The dosing ways of blocking or decreasing STING signaling activation are as follows: injection, oral, gene gun method, bacteria carrying plasmid DNA, adenovirus carrying target DNA or target gene encoding protein, electroporation, nasal drug delivery, pulmonary drug delivery, transdermal drug delivery, drug delivery within the tumor, and intracranial drug delivery.

### BRIEF DESCRIPTION OF THE DRAWINGS

FIG. 1 is a schematic diagram of prevention or treatment of AD by blocking or decreasing STING (TMEM173) signaling activation.

FIG. 2 is a collection of charts providing experimental results data in one embodiment.

FIG. 3 is a collection of charts providing experimental results data in one embodiment.

FIG. 4 is a collection of charts providing experimental results data in one embodiment.

FIG. 5 is a collection of charts providing experimental results data in one embodiment.

FIG. 6 is a collection of charts providing experimental results data in one embodiment.

### DETAILED DESCRIPTION

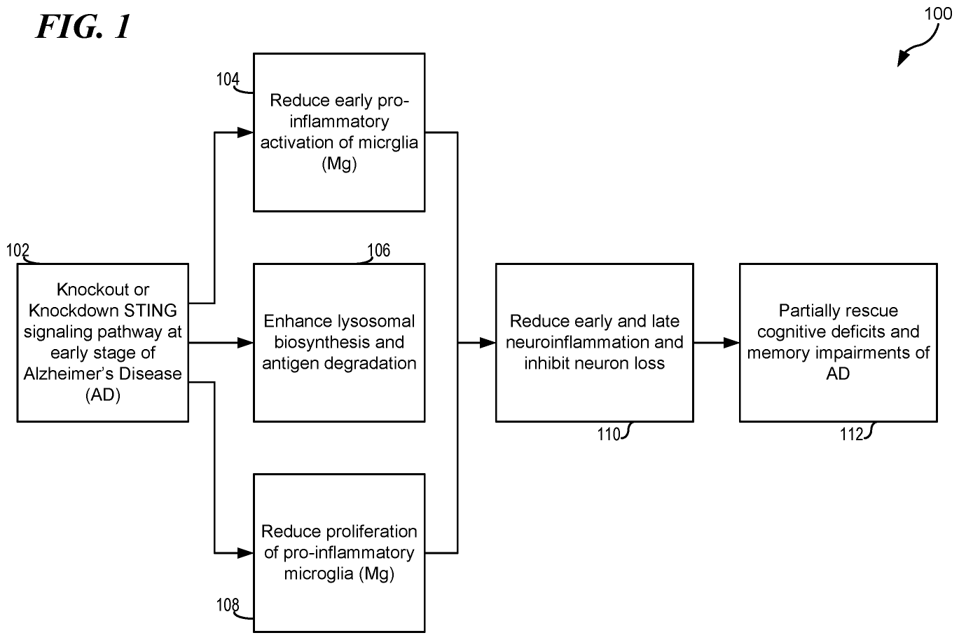

Referring to FIG. 1, presented is a schematic diagram of prevention or treatment of AD by blocking or decreasing STING (TMEM173) signaling activation is illustrated.

As shown in block 102, one embodiment includes first comprises knockout or knockdown of a STING signaling pathway at an early stage of Alzheimer's Disease (AD).

As shown in block 104, the illustrated embodiment includes reducing early pro-inflammatory activation of microglia (Mg).

As shown in block 106, the illustrated embodiment includes enhancing lysosomal biosynthesis and antigen degradation of microglia (Mg).

As shown in block 108, the illustrated embodiment includes reducing proliferation of pro-inflammatory microglia (Mg).

In one embodiment, the actions depicted in blocks 104, 106, and 108 are performed in any order with substantially overlapping execution times. In a preferred embodiment, the actions depicted in blocks 104, 106, and 108 are performed simultaneously. In one embodiment, actions performed simultaneously do not require strictly contemporaneous start and end times. In one embodiment, actions performed simultaneously can vary in execution times and such actions are performed simultaneously if the execution start and end times at least partially overlap for a majority of the actions.

As shown in block 110, the illustrated embodiment next reduces early and late neuroinflammation and inhibits neuron loss. Generally, the action depicted in block 110 occurs after the completion of active efforts in the actions depicted in blocks 104, 106, and 108.

As shown in block 112, the illustrated embodiment next partially rescues cognitive deficits and memory impairments of AD.

Experimental approaches confirmed the validity of the embodiments disclosed herein and in particular the embodiments as recited in the claims below.

Mouse Sting gene (Tmem173 gene) knockout and STING/Sting small molecule antagonist C-178 drug treatment in AD mouse models. The Tmem173−/−C57BL/6J mouse model and the 5XFAD B6.Cg mouse model were purchased from The Jackson Laboratory, USA. Four genotypes of NonTg, Tmem173−/−×NonTg, Tmem173−/−×5XFAD and 5XFAD were produced by hybridization of F1 and F2 generations. Participants were aged for 4.5 months old (4.5 mo) and 8.5 months old (8.5 mo), respectively. In the drug treatment study, the 3.5 months old (3.5 mo) NonTg and the 3.5 months old (3.5 mo) 5XFAD mouse models were treated with STING/Sting small molecule antagonist C-178 (6). Each dose of 375 nmol C-178 (Cayman Chemical, 329198-87-0) which was dissolved in 100 μL corn oil was intraperitoneally injected daily for 30 consecutive days.

Morris water maze was used to evaluate the cognitive ability of AD mouse models. Morris water maze were installed normally: a round white pool, 1 m in diameter and 50 cm high, water temperature of 23±2° C. Four equidistant points, N, E, S and W, were marked on the wall of the pool as the starting points, and the pool was divided into four quadrants. A platform was placed in the center of a quadrant, and the distance between the platform and the center of the pool was equal. The platform was below 1 cm of water and not visible. The reference cues were posted around the pool. The training lasted for 6 days, and each training time was 60 seconds. It was repeated 4 times a day at a certain time. Briefly, the mouse models were placed in water from 4 starting points at each time, then rested for 10 seconds after the mouse models found the platform or were guided to find the platform. Then the mouse models were dried and the training was repeated 20 minutes later. The tests were conducted 24 hours after the completion of the training. After withdrawing the platform, the mouse models were placed from the third quadrant into the water and recorded the swimming paths for 180 seconds. Time spent in the third quadrant and times through platform positions were recorded. One-way ANOVA statistics in Microsoft EXCEL software were used to analyze the data. The FIG. 2 revealed that blocking or decreasing Sting signaling activation can improve cognitive ability of the AD 5XFAD mouse models.

Quantitative RT-PCR (qRT-PCR) was used to determine neuroinflammation in AD mouse models. Tissue samples from the entorhinal cortex or the prefrontal lobe were fully dissolved in TRizol (Invitrogen, 15596018). The tissue samples were thoroughly ground in 1.5 mL tubes with RNase/Dnase-free pestle. Total RNA was purified by ZYMO Direct-Zol RNA Microprep Kits (ZYMO, R2063) and cDNA was synthesized by PrimeScript RT Master Mix (TaKaRa, RR036A). The cDNA was then preamplified with PerfeCTa PreAmp SuperMix (Quantabio, PN95146) according to the manufacturer's steps. The qPCR reactions were performed on Bio-Rad CFX Connect Real-time PCR detection system using PowerUp SYBR Green Master Mix (Applied Biosystems, A25742) with ROX reference dye (Invitrogen, #12223012). There were 8 samples from individual mouse models designed for each genotype and significance was evaluated by Student's t-Test in Microsoft EXCEL software. All RNA experiments were conducted in RNase/DNase-free environment to avoid RNA degradation and DNA contamination. The FIG. 3 revealed that blocking Sting signaling activation reduces neuroinflammation in the AD 5XFAD mouse models.

The brain cells (Mg, neuron, and astrocyte) were sorted by Fluorescence-Activated Cell Sorting (FACS) according to references (7), and the cell numbers of Mg and neuron were determined by FACS data analysis. Briefly, immediately after perfusion with cold PBS, the cortex and hippocampus of mouse models were separated and placed in cold DPBS (Gibco, 14190144). Then Accutase (Innovative Cell Technologies, NC9839010) was used to dissociate brain cells, and cell debris was removed by Debris Removal Solution (Miltenyi 130-109-398). The cells were fixed with 50% iced ethanol, and were washed twice by DPBS before antibody immunostaining. The fixed cells were labeled with antibodies for 20 min on ice, and sorted by BD FACSAria Fusion flow cytometry. The following antibodies and reagents were used for cell staining: Alexa Flour 488-conjugated anti-NeuN (Millipore, MAB377X, 1:1000), PE-conjugated anti-GFAP (BD Biosciences, 561483, 1:50), APC-conjugated anti-CD11b (BD Biosciences, 561690, 1:250), and DAPI (Invitrogen, D21490, 5 ng/mL). The cells were directly sorted into TRizol (Invitrogen, 15596018) and fully dissolved. There were 8 samples from individual mouse models designed for each genotype. All tissue and cell experiments were performed in ice baths and in endotoxin-free solutions at 4° C. The flow cytometry raw data were analyzed by FlowJo v10.6.1 to determine cell numbers. The significance was evaluated by Student's t-Test in Microsoft EXCEL software and the figures were generated by GraphPad Prism 8. The FIG. 4 revealed that blocking Sting signaling activation reduces microglia proliferation and neural loss in the AD 5XFAD mouse models.

Whole transcriptomics RNAseq library were prepared with rRNA depletion method. Briefly, 1,5000 sorted CD11b+myloid cells were fully lysed in TRizol (Invitrogen, 15596018) and total RNA were purified by Direct-zol RNA Microprep Kits (ZYMO, R2063). The purified total RNA was directly used for rRNA depletion and RNAseq library preparation using TaKaRa SMARTer Stranded Total RNA-Seq Kit v2-Pico Input Mammalian (TaKaRa, 634412). Total RNA and RNAseq library were quantified by Qubit 3.0 Fluorometer. The RNA integrity and cDNA fragments were analyzed by AATI Fragment Analyzer. The libraries were multiplexed and then sequenced on Illumina Nextseq 500 (Illumina) to generate 33M of single end 75 base pair reads per library. The RNAseq reads were aligned to Mus musculus UCSC mm10 (RefSeq gene annotation) using STAR aligner. Differential gene expression was performed by rlog Transformation function from DESeq2. Gene regulation significance was assessed with one-way ANOVA followed by Bonferroni's post hoc test. The significant regulations (adj. P.Val<0.05) were selected for analysis: (1) Gene regulation statistics (Venn Diagrams) was calculated by FunRich: Functional Enrichment analysis tool (8). (2) Microglia subgroup (DAM, Neurodegeneration, Interferon, Proliferation, LPS-Related) markers or modules analysis were performed according to previous publications (9). (3) Canonical Pathway, Upstream Analysis, Mechanistic Network of Upstream Regulators, Regulator Effect Network, and Causal Network were performed by Ingenuity Pathways Analysis (IPA, QIAGEN) pathway analysis. Putative upstream Cytokines, Transcriptional factors, Receptors, and Z-Score evaluation of activation or inhibitions of Canonical Pathways (Cellular Immune and Cytokine Signaling) were listed for pathway comparison. (4) KEGG and GO functional category enrichment statistics were performed by DAVID v6.8 (10). (5) FPKM (fragments per kilobase of exon model per million reads mapped) of major gene regulations were plotted by GraphPad Prism 8. There were 6 samples from individual mouse models designed for each genotype. The FIG. 5 revealed that blocking Sting signaling activation reduces microglia early pro-inflammatory activation in the AD 5XFAD mouse models.

Protease activity marker DQ Ovalbumin (DQ-OVA, Molecular Probes, D12053) assay was used to evaluate lysosomal biosynthesis and protein antigen degradation of Mg cells. Briefly, the primary Mg which were purified from the 5XFAD mouse models using CD11b MicroBeads (Miltenyi Biotec, 130-093-634) were cultured in EMEM medium (Gibco, 670086) with 10% heat-inactived FBS (Gibco, 26140079) and 1% PS (Gibco, 15140122) on 12-well cell culture plates for 24 hr. The test cells were pre-treated by STING/Sting antagonist C-178 (Cayman Chemical, 329198-87-0) for 1 hr, while the control cells were pre-treated by equivalent DMSO solvent for 1 hr, in 37° C. cell incubator. The DQ-OVA was diluted at 5 mg/mL in PBS as stock. For each treated well, the pre-treated cells were treated with 5 μg/mL DQ-OVA for 30 min and immediately transferred onto ice-surface, then were further stained with Lysotracker (Invitrogen) for 15 min on ice. The cells in each well were divided into 2 equal samples. One sample was analyzed by BD LSRFortessa flow cytometry, while another sample was fully dissolved in TRizol (Invitrogen). The concentration and treatment procedure of STING/Sting antagonist C-178 (2 μM pre-treated for 1 hr), DQ-OVA (5 μg/mL treated for 30 min) were designed according to manufacturer's protocols and references. The gene regulations of lysosomal biosynthesis were also evaluated by qRT-PCR in this cell treatment models according to the above-mentioned procedures (16). For each treatment, there were 3 biological replicates (wells) designed and significance was evaluated by Student's t-Test in Microsoft EXCEL software. The flow cytometry data were analyzed by FlowJo v10.6.1 software and Microsoft EXCEL software. And the qRT-PCR figures were generated by GraphPad Prism 8. The FIG. 6 revealed that blocking or decreasing Sting signaling activation enhanced lysosomal biosynthesis and protein antigen degradation in the Mg cells of the AD 5XFAD mouse models.

Additional details and understanding may be achieved by reference to experimental data results, described in more detail below.

FIG. 2 presents a collection of charts providing selected experimental data results in one embodiment. As illustrated, Morris water maze revealed that blocking or decreasing Sting signaling activation improved cognitive ability of the AD 5XFAD mouse models. (2A-2F) Blocking Sting signaling activation by gene knockout partially rescued cognitive deficits and memory impairments in the 4.5 months old (4.5 mo) (2A-2C) and in the 8.5 months (8.5 mo) old (2D-2F) 5XFAD mouse models, but did not affect swimming speeds. (2G-2I) Decreasing Sting signaling activation by STING/Sting small molecule antagonist (C-178) partially rescued cognitive deficits and memory impairments in the 4.5 months old (4.5 mo) 5XFAD mouse models, but did not affect swimming speeds. (* P<0.05, ** P<0.01, *** P<0.001)

FIG. 3 presents a collection of charts providing selected experimental data results in one embodiment. As shown, blocking Sting signaling activation reduced neuroinflammation in the AD 5XFAD mouse models. (3A-3F) Blocking Sting signaling activation by gene knockout significantly downregulated the gene expressions of the major pro-inflammatory factors, Ifna4, Ifnb1, Il1a, Il1b, I16, and Tnf. (3G-3H) Blocking Sting signaling activation by gene knockout also significantly downregulated the gene expressions of the major anti-inflammatory factors, Il10 and I14. (3I-3K) Blocking Sting signaling activation by gene knockout did not influence the Trem2 signaling activation. There were 8 samples from individual mouse models designed for each genotype. (* P<0.05, ** P<0.01, *** P<0.001)

FIG. 4 presents a collection of charts providing selected experimental data results in one embodiment. As shown, blocking Sting signaling activation reduced microglia proliferation and neural loss in the AD 5XFAD mouse models. (4A) The microglia (Mg), neuron, and astrocyte were sorted by Fluorescence-Activated Cell Sorting (FACS) using anti-CD11b, anti-NeuN, and anti-GFAP antibody staining, respectively. (4B) Blocking Sting signaling activation by gene knockout significantly reversed the Mg proliferation in the 9 months old (9 mo) 5XFAD mouse models. (4C) Blocking Sting signaling activation by gene knockout significantly reversed the neuron loss in the 9 months old (9 mo) 5XFAD mouse models. There were 8 samples from individual mouse models designed for each genotype. (* P<0.05, ** P<0.01, *** P<0.001)

FIG. 5 presents a collection of charts providing selected experimental data results in one embodiment. As shown, blocking Sting signaling activation reduced microglia early pro-inflammatory activation in the AD 5XFAD mouse models. (5A-5B) Venn Diagrams revealed that blocking Sting signaling activation by gene knockout reversed the 140 gene upregulations in the Mg of the 2 months old (2 mo) 5XFAD mouse models (5A) and reversed the 100 gene upregulations in the Mg of the 9 months old (9 mo) 5XFAD mouse models (5B). (5C) Blocking Sting signaling activation by gene knockout downregulated gene expression of Interferon (Ifn) and the Interferon-Stimulated Genes (ISGs). (5D) Blocking Sting signaling activation by gene knockout reversed the activation of ‘Neuroinflammation Signaling Pathway’ and ‘TREM1 Signaling’ pathways in the Mg of the 2 months old (2 mo) 5XFAD mouse models. (5E) Blocking Sting signaling activation by gene knockout downregulated a series of gene regulations of the pro-inflammatory factors. (5F) Blocking Sting signaling activation by gene knockout reversed the gene upregulations of the major pro-inflammatory factors (Tnf and Ifng) in the Mg of the 2 months old (2 mo) 5XFAD mouse models. There were 6 samples from individual mouse models designed for each genotype. This indicated that the early pro-inflammatory activation of the Mg in the AD 5XFAD mouse models were partially blocked by Sting gene knockout. (* P<0.05, ** P<0.01, *** P<0.001)

FIG. 6 presents a collection of charts providing selected experimental data results in one embodiment. As shown, blocking or decreasing Sting signaling activation enhanced lysosomal biosynthesis and protein antigen degradation in the 5XFAD Mg cells. (6A) Transcriptomics pathway enrichment analysis revealed that blocking Sting signaling activation by gene knockout significantly (logP=−2) enhanced ‘Lysosome pathway’ in the Mg of the 2 months old (2 mo) 5XFAD mouse models. (6B-6C) Representative figures of the flow cytometry analysis of the lysosomal biosynthesis (Lysotracker) and the protein antigen degradation (DQ-OVA) of the Mg from the 3 months old (3 mo) 5XFAD mouse models (6B) and the Mg from the STING/Sting antagonist C-178 treated Mg from the 3 months old (3 mo) 5XFAD mouse models (6C). (6D) The qRT-PCR analysis revealed that decreasing Sting signaling activation by STING/Sting antagonist C-178 treatment significantly reduced Ifn-ISG expression (Ifna4, Ifnb1, Irf7, and Isg15) and enhanced lysosomal biosynthesis (Tfeb, Ctsd, Ctsb, and Lamp1). The fold changes were the ratios of the expression levels of the C-178 treated 5XFAD Mg to the expression levels of the DMSO treated 5XFAD Mg control. All the regulations of the targets were significant (P<0.05, or P<0.01). (6E-6G) Statistics of the flow cytometry analysis (6B-6C) of the lysosomal biosynthesis (Lysotracker) and the protein antigen degradation (DQ-OVA) of the C-178 treated Mg. Decreasing Sting signaling activation by STING/Sting antagonist C-178 treatment significantly enhanced the lysosomal biosynthesis (Lysotracker) and the protein antigen degradation (DQ-OVA) (Q2 quadrant) in the Mg from the 3 months old (3 mo) 5XFAD mouse models.

### REFERENCES

1. A. C. Cuello, Early and Late CNS Inflammation in Alzheimer's Disease: Two Extremes of a Continuum? Trends in pharmacological sciences 38, 956-966 (2017).
2. I. Benilova, E. Karran, B. De Strooper, The toxic Abeta oligomer and Alzheimer's disease: an emperor in need of clothes. Nature neuroscience 15, 349-357 (2012).
3. D. L. Burdette, K. M. Monroe, K. Sotelo-Troha, J. S. Iwig, B. Eckert, M. Hyodo, Y. Hayakawa, R. E. Vance, STING is a direct innate immune sensor of cyclic di-GMP. Nature 478, 515-518 (2011).
4. J. M. Taylor, Z. Moore, M. R. Minter, P. J. Crack, Type-I interferon pathway in neuroinflammation and neurodegeneration: focus on Alzheimer's disease. Journal of Neural Transmission 125, 797-807 (2018).
5. S. D. Mesquita, A. C. Ferreira, F. Gao, G. Coppola, D. H. Geschwind, J. C. Sousa, M. Correia-Neves, N. Sousa, J. A. Palha, F. Marques, The choroid plexus transcriptome reveals changes in type I and II interferon responses in a mouse model of Alzheimer's disease. Brain, behavior, and immunity 49, 280-292 (2015).
6. S. M. Haag, M. F. Gulen, L. Reymond, A. Gibelin, L. Abrami, A. Decout, M. Heymann, F. G. van der Goot, G. Turcatti, R. Behrendt, A. Ablasser, Targeting STING with covalent small-molecule inhibitors. Nature 559, 269-273 (2018).
7. K. Srinivasan, B. A. Friedman, J. L. Larson, B. E. Lauffer, L. D. Goldstein, L. L. Appling, J. Borneo, C. Poon, T. Ho, F. Cai, P. Steiner, M. P. van der Brug, Z. Modrusan, J. S. Kaminker, D. V. Hansen, Untangling the brain's neuroinflammatory and neurodegenerative transcriptional responses. Nature communications 7, 11295 (2016).
8. M. Pathan, S. Keerthikumar, C. S. Ang, L. Gangoda, C. Y. Quek, N. A. Williamson, D. Mouradov, O. M. Sieber, R. J. Simpson, A. Salim, A. Bacic, A. F. Hill, D. A. Stroud, M. T. Ryan, J. I. Agbinya, J. M. Mariadason, A. W. Burgess, S. Mathivanan, FunRich: An open access standalone functional enrichment and interaction network analysis tool. Proteomics 15, 2597-2601 (2015).
9. B. A. Friedman, K. Srinivasan, G. Ayalon, W. J. Meilandt, H. Lin, M. A. Huntley, Y. Cao, S. H. Lee, P. C. G. Haddick, H. Ngu, Z. Modrusan, J. L. Larson, J. S. Kaminker, M. P. van der Brug, D. V. Hansen, Diverse brain myeloid expression profiles reveal distinct microglial activation states and aspects of Alzheimer's Disease not evident in mouse models. Cell reports 22, 832-847 (2018).
10. D. W. Huang, B. T. Sherman, R. A. Lempicki, Systematic and integrative analysis of large gene lists using DAVID bioinformatics resources. Nature Protocols 4, 44-57 (2009).

What is claimed is:

1. Blocking or decreasing the STING signaling activation in the preparation of products for the prevention or treatment of AD.

2. As described in claim 1, AD can be mild, moderate or severe AD.

3. As described in claim 1, STING signaling pathway refers to either of the cGAS-STING-TBK1-IRF3-IFN I cascade signaling pathway or the cGAS-STING-IKK-NFκB-IFN I cascade signaling pathway or both.

4. As described in claim 1, the methods for blocking or decreasing STING signaling activation refer to antibodies, siRNA, miRNA, antisense oligonucleotides, CRISPR/Cas9 gene editing products, antagonists, blockers, small molecule compounds that inhibit the function and/or activity of main molecules of STING signaling pathway, and one of or a combination of promoter elements or expression vectors, which can downregulate the gene expression levels, the mRNA expression levels, or the protein expression levels of the major molecules of the STING signaling pathway.

5. As described in claim 1, the symptoms of blocking or decreasing STING signaling activation include one or more of the following groups: the decreased early pro-inflammatory activation of Mg cells, the decreased proliferation of the pro-inflammatory Mg cells, the enhanced lysosomal synthesis and the protein antigen degradation of Mg cells, the decreased neuron loss, the decreased AD neuroinflammation, and the improved cognitive ability.

6. Blocking or decreasing STING signaling activation targets human STING signaling pathway.

7. The products include medicine, food, and health care products and their compositions.

8. The dosing ways of blocking or decreasing the STING signaling activation are as follows: injection, oral, gene gun method, bacteria carrying plasmid DNA, adenovirus carrying target DNA or target gene encoding protein, electroporation, nasal drug delivery, pulmonary drug delivery, transdermal drug delivery, drug delivery within the tumor, and intracranial drug delivery.
